(19) United States
(12) Patent Application Publication
(10) Pub. No.: US 2021/0223264 A1
(43) Pub. Date: Jul. 22, 2021

(54) DIAGNOSTIC METHOD(S) FOR DETECTING AND TREATING POST-INFARCT MYOCARDIUM REMODELING AND DIFFUSE MYOCARDIAL FIBROSIS

(71) Applicant: Siemens Healthcare Diagnostics Inc., Tarrytown, NY (US)

(72) Inventors: Luis LASALVIA, White Plains, NY (US); Antonio BERRUEZO SANCHEZ, Barcelona (ES); Manuel MORALES-RUIZ, Barcelona (ES); Jose TOMAS ORTIZ PEREZ, Barcelona (ES); Rosario JESUS PEREA PALAZON, Barcelona (ES); Aurea MIRA VALLET, BARCELONA (ES)

(73) Assignee: Siemens Healthcare Diagnostics Inc., Tarrytown, NY (US)

(21) Appl. No.: 16/096,696

(22) PCT Filed: Apr. 25, 2017
(86) PCT No.: PCT/US17/29459

§ 371 Date: Oct. 25, 2018

Related U.S. Application Data
(60) Provisional application No. 62/327,278, filed on Apr. 25, 2016.

(51) Int. Cl. G01N 33/68

(52) U.S. Cl. CPC G01N 33/6893; G01N 2800/324; G01N 2333/4724; G01N 2800/52

(57) ABSTRACT

Diagnostic method(s) for detecting and treating post-infarct myocardial remodeling and diffuse myocardial fibrosis.

### CROSS REFERENCE TO RELATED APPLICATION(S)/INCORPORATION BY REFERENCE STATEMENT

The present application claims the benefit of U.S. Provisional Application No. 62/327,278, filed Apr. 25, 2016. The entire contents of the above-referenced provisional application are hereby expressly incorporated by reference.

### STATEMENT REGARDING FEDERALLY SPONSORED RESEARCH OR DEVELOPMENT

Not Applicable.

### TECHNICAL FIELD

The presently disclosed and/or claimed inventive concept(s) generally relates to the method(s) for detecting and/or treating post-infarct myocardium remodeling. More particularly, but not by way of limitation, the presently disclosed and/or claimed inventive concept(s) relates to methods of detecting and/or treating post-infarct myocardium remodeling via the correlation of serum biomarkers present in survivors of myocardial infarction, including, but not limited to, ST-elevational myocardial infarction (STEMI) and via targeting the expression and/or production of such serum biomarkers.

### BACKGROUND

Myocardium remodeling, including, without limitation, left ventricular (LV) remodeling, after a patient suffers a myocardial infarction episode, including, but not limited to, STEMI, leads to the development of ischemic LV dysfunction, a process characterized by structural and functional alterations involving the myocardium of a patient, and which is related to patient prognosis and survival. LV-remodeling begins early after a STEMI episode, the extent of which is dependent on the size of the infarct and the phenotypic transformation of cardiac cells, including, but not limited to, cardiac myocytes and cardiac non-myocyte cells, as well as the changes in the composition of the cardiac extracellular matrix (ECM).

The ECM primarily comprises, by way of example only, elastin, glycoproteins, glycosaminoglycans, and collagen fibrils that are generally exposed to homeostatic synthesis and degradation control mediated by the activity of cardiac fibroblasts and extracellular proteases. However, in pathological conditions, such as following a STEMI episode, alterations in the composition of the ECM lead to diffuse myocardial fibrosis (DMF) in the remote myocardium, as well as replacement fibrosis in the infarcted myocardium. The exacerbation of these pathological features plays a major role in the development of cardiac wall stiffness, arrhythmia, heart failure and, in some cases, sudden death.

It is well understood in the art that cardiac fibrosis is an independent predictor of major adverse cardiac events. Cardiac magnetic resonance (CMR) is emerging as a modality that enables non-invasive evaluation of the myocardial interstitial space through measurements of the extracellular volume (ECV) via T1 mapping techniques. It is commonly known in the art that ECV is closely correlated to reflect the degree of histologic DMF. Late gadolinium enhancement cardiac resonance (LGE-CMR) following the administration to a patient of a gadolinium-based contrast agent(s) (GBCA) is the reference standard for noninvasive detection of focal fibrosis. However, the detection of DMF cannot be evaluated by LGE-CMR. Instead, DMF in the myocardial tissue can only be detected by using endomyocardial biopsy (EMB), which is known in the art to be highly invasive and associated with a risk of procedural complications.

There is currently a need to establish non-invasive methods for diagnosing and predicting the degree (and significance) of a patient's ECV expansion following an infarction episode, including, without limitation, a STEMI episode. The presently disclosed and/or claimed inventive concept(s) are directed to such method(s), including, without limitation, evaluating the relationship between early serum biomarkers (including, without limitation, galectin-3 (Gal3) and B-type natriuretic peptide (BNP)), the levels of which are measured very soon after a myocardial infarction episode, including, without limitation at various times during the first seven (7) days following a STEMI episode, and correlating these levels to the degree of post-infarct remodeling of the remote myocardium as evaluated by measuring the ECV via CMR at one hundred and eighty (180) days following a STEMI episode. It is to such method(s) that the presently disclosed and/or claimed inventive concept(s) is directed.

### BRIEF DESCRIPTION OF THE DRAWINGS

FIG. 1 graphically depicts a flow chart that details the selection process for patient inclusion for the data collected.

FIGS. 2A-2C show the quantification of extracellular volume fraction (ECV) from pre- and post-contrast T1 mapping in a patient with percutaneous coronary intervention one hundred and eighty (180) days prior for right coronary artery occlusion. For FIGS. 2A-2C, ECV was calculated in the T1 maps with regions of interest drawn pre- and 15 minutes' post-contrast with the equation: ECV=(1−Hematocrit)×(ΔR1myocardium/ΔR1blood); where ΔR1 is (1/T1 precontrast−1/T1 post-contrast). As shown in FIGS. 2A-2C ECV in the remote myocardium was 29.5% (slightly elevated, reflecting diffuse fibrosis).

FIG. 2A more specifically shows a late gadolinium enhancement image showing an inferior scar in accordance with the presently disclosed and/or claimed inventive concept(s).

FIG. 2B more specifically shows a quantification of ECV from pre-contrast T1 mapping in accordance with the presently disclosed and/or claimed inventive concept(s).

FIG. 2C more specifically shows a quantification of ECV post-contrast T1 mapping in accordance with the presently disclosed and/or claimed inventive concept(s).

FIG. 3A shows a correlation plot illustrating the correlation between ECV at one hundred and eighty (180) days post-STEMI, BNP at seven (7) days post-STEMI, galectin-3 at seven (7) days post-STEMI, and the rest of the imaging/laboratory parameters determined in patients post-STEMI. The bottom bar indicates the equivalence between the code and the value of the correlation coefficient (r).

FIG. 3B is a graphical representation of galectin-3 (left graph) and BNP (right graph) levels measured in patients at seven (7) days post-STEMI compared with the ECV score of the patients measured one hundred eighty (180) days post-STEMI.

FIG. 4 depicts a classification and regression tree (CART) decision tree model showing the direct correlation between the level(s) of a galectin-3 biomarker as a predictor to stratify patients with high ECV values (shown in FIG. 4 as values greater than 28.5%) after one hundred eighty (180) day post-STEMI. In FIG. 4, the optimal cutoff value for galectin-3 was 10.15 ng/mL (with a patient sample size of 26 patients). The rectangular boxes at the bottom of the tree provide a proportion scale to assess the accuracy of the CART algorithm to predict patients with ECV values of ≤28% or >28%.

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the articles, compositions, and/or methods disclosed herein can be made and executed without undue experimentation in light of the present disclosure. While the articles, compositions, and methods of the inventive concept(s) have been described in terms of particular embodiments, it will be apparent to those of skill in the art that variations may be applied to the articles, compositions and/or methods and in the steps or in the sequence of steps of the methods described herein without departing from the concept, spirit, and scope of the inventive concept(s). All such similar substitutes and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

The term “sample” as used herein will be understood to include any type of biological sample that may be utilized in accordance with the presently disclosed and/or claimed inventive concept(s). Examples of fluidic biological samples that may be utilized include, but are not limited to, whole blood or any portion thereof (i.e., plasma or serum), urine, saliva, sputum, cerebrospinal fluid (CSF), skin, intestinal fluid, intraperitoneal fluid, cystic fluid, sweat, interstitial fluid, extracellular fluid, tears, mucus, bladder wash, semen, fecal, pleural fluid, nasopharyngeal fluid, combinations thereof, and the like.

The term “patient” includes human and veterinary subjects. In certain embodiments, a patient is a mammal. In certain other embodiments, the patient is a human. “Mammal” for purposes of treatment refers to any animal classified as a mammal, including human, domestic and farm animals, nonhuman primates, and zoo, sports, or pet animals, such as dogs, horses, cats, cows, etc. In one non-limiting embodiment of the presently disclosed and/or claimed inventive concept(s), the patient(s) is/are human subject(s) who has/have suffered at least one myocardial infarction episode.

The term “purified” as used herein means at least one order of magnitude of purification is achieved compared to the starting material or of the natural material, for example but not by way of limitation, two, three, four, or five orders of magnitude of purification of the starting material or of the natural material. Thus, the term “purified” as utilized herein does not necessarily mean that the material is 100% purified, and therefore such term does not exclude the presence of other material(s) present in the purified composition.

As used herein, “substantially pure” means an object species is the predominant species present (i.e., on a molar basis it is more abundant than any other individual species in the composition), and particularly a substantially purified fraction is a composition wherein the object species comprises at least about 50 percent (on a molar basis) of all macromolecular species present. Generally, a substantially pure composition will comprise more than about 80 percent of all macromolecular species present in the composition, such as more than about 85%, 90%, 95%, and 99%. In one embodiment, the object species is purified to essential homogeneity (contaminant species cannot be detected in the composition by conventional detection methods) wherein the composition consists essentially of a single macromolecular species.

“Treatment” refers to both therapeutic treatment and prophylactic or preventative measures. Those in need of treatment include, but are not limited to, individuals already having a particular condition or disorder as well as individuals who are at risk of acquiring a particular condition or disorder (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect. In one non-limiting embodiment, the therapeutic composition(s) comprise or consist of at least one compound for treating diffuse myocardial fibrosis, including, but not limited to angiotensin receptor blockers, beta blockers, calcium channel blockers, renin-angiotensin system (RAS) inhibitors, such as, by way of example only, angiotensin-converting-enzyme (ACE) inhibitors, Ang II receptor blockers (ARBs), direct renin inhibitors, and aldosterone antagonists, transforming growth factor beta (TGF-beta) inhibitors and endothelin (ET) inhibitors, histone deacetylases (HDACs) inhibitors, ivabradine, and additional agents, including, but not limited to, diltiazem, tadalafil, isosorbide dinitrate and hydralazine, erythropoietin, cyclosporine, thalidomide, and anti-inflammatory drugs impacting cytokines, TD139 galectin-3 inhibitor, or combinations thereof.

Administering a therapeutically effective amount or prophylactically effective amount is intended to provide a therapeutic benefit in the treatment, reduction in occurrence, prevention, or management of a disease and/or cancer. The specific amount that is therapeutically effective can be readily determined by the ordinary medical practitioner, and can vary depending on factors known in the art, such as the type of disease/cancer, the patient's history and age, the stage of disease/cancer, and the co-administration of other agents.

A “disorder” is any condition, including, without limitation, cardiac fibrosis and diffuse myocardial fibrosis, that would benefit from treatment with a therapeutic composition. This includes chronic and acute disorders or diseases including those pathological conditions which predispose the mammal to the disorder in question.

The term “effective amount” refers to an amount of a biologically active molecule or conjugate or derivative thereof sufficient to exhibit a detectable therapeutic effect without undue adverse side effects (such as toxicity, irritation and allergic response) commensurate with a reasonable benefit/risk ratio when used in the manner of the inventive concepts. The therapeutic effect may include, for example but not by way of limitation, inhibiting the growth of undesired tissue or malignant cells. The effective amount for a subject will depend upon the type of subject, the subject's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy”, and will be understood to mean that the patient in need of treatment is treated or given another drug for the disease in conjunction with the pharmaceutical compositions of the presently disclosed and/or claimed inventive concepts. This concurrent therapy can be sequential therapy, where the patient is treated first with one drug and then the other drug, or the two drugs can be given simultaneously.

The terms “administration” and “administering” as used herein will be understood to include all routes of administration known in the art, including but not limited to, oral, topical, transdermal, parenteral, subcutaneous, intranasal, mucosal, intramuscular, intraperitoneal, intravitreal and intravenous routes, including both local and systemic applications. In addition, the compositions of the presently disclosed inventive concepts (and/or the methods of administration of same) may be designed to provide delayed, controlled or sustained release using formulation techniques which are well known in the art.

As used herein, a “therapeutically effective amount” of the inhibitor or chemotherapeutic agent of the presently disclosed inventive concepts refers to an amount of a compound that is effective, upon single- or multiple-dose administration to the subject, e.g., a patient, at treating, inhibiting, mitigating, reducing, modulating, or otherwise affecting any of the disorders, diseases, or conditions described elsewhere herein, for example, orthostatic hypotension, or any other condition involving a disorder, disease or condition which involves autoantibodies to any of the receptors described herein.

The term “infarction” or “myocardial infarction” or “acute myocardial infarction” or “infarction episode” as used herein has the meaning(s) commonly known in the art, including, but not limited, the irreversible necrosis of heart muscle due to prolonged lack of oxygen resulting from ischemia. In one non-limiting embodiment of the presently disclosed and/or claimed inventive concept(s), the term infarction refers to ST-segment elevation myocardial infarction (STEMI).

The term “remodeling” or “cardiac remodeling” or “ventricular remodeling” as used herein will be understood to refer changes in the size, shape, structure, and/or function of a patient's heart, including, without limitation, changes in the size, shape, structure, and/or function of a patient's heart as a result of acute myocardial infarction, including, without limitation, STEMI. Remodeling, as used herein, further includes, but is not limited to, cardiomyopathy (including myocardial extracellular volume (ECV)), ventricular hypertrophy, ventricular dilation, cardiomegaly, cardiac pressure overload, and/or cardiac volume overload.

As used herein, “pharmaceutically acceptable” refers to those properties and/or substances, which are acceptable to the patient from a pharmacological/toxicological point of view including bioavailability and patient acceptance or to the manufacturing chemist from a physical-chemical point of view regarding composition, formulation, stability and isolatability. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically-acceptable carrier” as used herein means a pharmaceutically-acceptable material, composition, or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Examples of pharmaceutically acceptable carriers that may be utilized in accordance with the presently disclosed and/or claimed inventive concepts include, but are not limited to, PEG, liposomes, ethanol, DMSO, aqueous buffers, oils, DPPC, lipids, other biologically-active molecules, vaccine-adjuvants, and combinations thereof.

By “biologically active” is meant the ability to modify the physiological system of an organism. A molecule can be biologically active through its own functionalities, or may be biologically active based on its ability to activate or inhibit molecules having their own biological activity.

The term “electrode” as used herein refers to any type of conductor or medium that is capable of functioning in accordance with the presently disclosed inventive concept(s). Non-limiting examples of electrodes that fall within the scope of the presently disclosed and/or claimed inventive concept(s) include electrochemical cells comprising a plurality of electrodes. Exemplary electrochemical cell constructs include a two-electrode cell comprising one indicator electrode and one reference electrode, a two-electrode cell comprising one anode and one cathode, a three-electrode cell comprising one anode, one cathode and one reference electrode, and a four-electrode cell comprising two working electrodes, one counter electrode, and one reference electrode.

The term “probe” as used herein will be understood to refer to any type of affinity reagent that binds to a specific biomarker as described herein. Examples of probes include, but are not limited to, antibodies (or binding fragments or derivatives thereof), receptors, organic molecules, inorganic molecules, ligands, nucleic acids (including but not limited to, DNA, RNA, microRNA, mRNA, siRNA, etc.), peptides, polypeptides, proteins, epitopes, antigens, ligands, receptors, complexes, lipids, glycoproteins, glycolipids, glycosaminoglycans, carbohydrates, polycarbohydrates, glycoconjugates, and any combination or derivative thereof.

The term “biomarker” as used herein will be understood to refer to any target site on the surface of or inside of a cell that a probe can have affinity therefor and thus can bind to said moiety. The “biomarker” may be, for example but not by way of limitation, a nucleic acid, peptide, polypeptide, protein, epitope, antigen, ligand, receptor, complex (i.e., an MHC-peptide complex), lipid, glycoprotein, glycolipid, glycosaminoglycan, carbohydrate, polycarbohydrate, glycoconjugate, and any combination or derivative thereof. In one non-limiting embodiment of the presently disclosed and/or claimed inventive concept(s), the biomarker is selected from the group comprising or consisting of galectin-3 (Gal3) and B-type natriuretic peptide (BNP).

The terms “peptide”, “polypeptide” and “protein” are used herein to refer to a polymer of amino acid residues. The term “polypeptide” as used herein is a generic term to refer to native protein, protein fragments, or analogs of a polypeptide sequence. Hence, native protein, protein fragments, and analogs are species of the polypeptide genus.

The terms “polynucleotide”, and “nucleic acid” are used interchangeably. They refer to a polymeric form of nucleotides of any length, either deoxyribonucleotides or ribonucleotides, or analogs thereof. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The terms “isolated nucleic acid” and “isolated polynucleotide” are used interchangeably; a nucleic acid or polynucleotide is considered “isolated” if it: (1) is not associated with all or a portion of a polynucleotide in which the “isolated polynucleotide” is found in nature, (2) is linked to a polynucleotide to which it is not linked in nature, or (3) does not occur in nature as part of a larger sequence.

The term “antibody” is used in the broadest sense, and specifically covers monoclonal antibodies (including full length monoclonal antibodies), polyclonal antibodies, multispecific antibodies (e.g., bispecific antibodies), and antibody fragments so long as they exhibit the desired biological activity. Thus, the terms “Antibody” or “antibody peptide(s)” refer to a full length immunoglobulin molecule (i.e., an intact antibody), or a binding fragment thereof that competes with the intact antibody for specific antigen binding. Binding fragments may be produced by recombinant DNA techniques, or by enzymatic or chemical cleavage of intact antibodies. Binding fragments include Fab, Fab′, F(ab′)2, Fv, scFv, disulfide linked Fv, Fd, diabodies, single-chain antibodies, single domain antibodies (such as but not limited to, NANOBODIES®) and other antibody fragments that retain at least a portion of the variable region of an intact antibody. See, e.g., Hudson et al. (Nature Med., 9:129-134 (2003)). The antibody can be of any type or class (e.g., IgG, IgE, IgM, IgD, and IgA) or sub-class (e.g., IgG1, IgG2, IgG3, IgG4, IgA1, and IgA2).

The term “antigen binding fragment” or “antigen-binding portion” of an antibody, as used herein, refers to one or more fragments of an antibody that retain the ability to bind to an antigen. The antigen-binding function of an antibody can be performed by fragments of an intact antibody. Examples of binding fragments encompassed within the term “antigen-binding fragment” of an antibody include but are not limited to, Fab, Fab′, F(ab′)2, Fv, scFv, disulfide linked Fv, Fd, diabodies, single-chain antibodies, single domain antibodies (such as but not limited to, NANOBODIES®), isolated CDRH3, and other antibody fragments that retain at least a portion of the variable region of an intact antibody. These antibody fragments are obtained using conventional recombinant and/or enzymatic techniques and are screened for antigen binding in the same manner as intact antibodies.

While immunoassays are primarily discussed herein, a person having ordinary skill in the art should readily understand that the presently disclosed and claimed inventive concept(s) are not strictly limited to immunoassays and may include, by way of example and not by limitation, nucleic acid capture assays and serology-based assays. Immunoassays, including radioimmunoassays and enzyme-linked immunoassays, are useful methods for use with the presently claimed and disclosed inventive concepts. A variety of immunoassay formats, including, for example, competitive and non-competitive immunoassay formats, antigen/analyte capture assays and two-antibody sandwich assays can be used in the methods of the invention. Enzyme-linked immunosorbent assays (ELISAs) can be used in the presently claimed and disclosed inventive concepts, as well. In the case of an enzyme immunoassay, an enzyme is typically conjugated to a second antibody, generally by means of glutaraldehyde, periodate, hetero-bifunctional crosslinking agents, or biotin-streptavidin complexes. As will be readily recognized, however, a wide variety of different conjugation techniques exist which are readily available for use with the presently disclosed and claimed inventive concept(s) to one skilled in the art.

The term “epitope” includes any protein determinant capable of specific binding to an immunoglobulin or T-cell receptor. In certain embodiments, an epitope is a region of an antigen that is specifically bound by an antibody. Epitopic determinants usually include chemically active surface groupings of molecules such as amino acids, sugar side chains, phosphoryl, or sulfonyl groups. In certain embodiments, an epitope may have specific three dimensional structural characteristics (e.g., a “conformational epitope”), as well as specific charge characteristics.

The term “nanoparticle” as used herein refers to a particle having dimensions of from about 1 to about 5000 nanometers, and having any size, shape or morphology. The nanoparticles utilized in accordance with the presently disclosed and/or claimed inventive concept(s) may be naturally occurring, commercially available nanoparticles, or the nanoparticles may be synthesized for use in accordance with the presently disclosed and/or claimed inventive concept(s), as described herein below and as known in the art. Particular examples of nanoparticles that may be utilized in accordance with the presently disclosed and/or claimed inventive concept(s) include, but are not limited to, poly(lactic-co-glycolic) acid (PLGA) nanoparticles, poly lactic acid (PLA) nanoparticles, Chitosen nanoparticles, liposomes, and derivatives or combinations thereof.

As used herein, the terms “label” or “labeled” refers to incorporation of a detectable marker, e.g., by attachment of a fluorescent, enzymatic or colorimetric label or incorporation of a radiolabeled amino acid. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent, biotinyl groups, predetermined polypeptide epitopes recognized by a secondary reporter (e.g., leucine zipper pair sequences, binding sites for secondary antibodies, metal binding domains, epitope tags). In some embodiments, labels are attached by spacer arms of various lengths to reduce potential steric hindrance.

The terms “label”, “detectable marker” and “detection moiety” are used interchangeably herein.

A fluorophore may be employed in the methods of the presently disclosed and/or claimed inventive concept(s) and detected via any of numerous colorimetric and fluorescence detection methods. Depending on the application and purpose, such methods include, but are not limited to, absorbance spectroscopy, fluorescence spectroscopy, fluorescence activated cytometry (FACS), fluorescence microscopy, fluorescence resonance energy transfer (FRET), and the like.

Various types of fluorophores, depending on the application and purpose, may be employed in accordance with the presently disclosed and/or claimed inventive concept(s). Examples of suitable fluorophores are described herein below. Examples of suitable fluorophores are described herein below. Other examples are given in U.S. Pat. Nos. 7,465,747 and 7,955,859, issued to Matsumoto et al. on Dec. 16, 2008 and Jun. 7, 2011, respectively; and US Publication No. US2007/0026407, published Feb. 1, 2007 (the entire contents of which are expressly incorporated herein by reference in their entirety).

Ample guidance regarding fluorophore selection, methods of linking fluorophores to various types of molecules, and methods of use thereof is available in the literature of the art [for example, refer to: Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, U K. (2001); U.S. Pat. No. 6,350,466 to Targesome, Inc.]. Therefore, no further description is considered necessary.

The terms “substantial increase” and “substantial decrease”, as well as grammatical equivalents thereof, will be understood herein to refer to at least a 12% increase or decrease, such as at least a 30% increase or decrease, at least a 50% increase or decrease, at least a 75% increase or decrease, or at least a 90% increase or decrease.

The phrase “providing a biological sample” as used herein refers to obtaining a biological sample for use in methods described and claimed herein. Most often, this will be done by removing a sample of cells from a patient, but can also be accomplished by using previously isolated cells (e.g., isolated by another person, at another time and/or for another purpose). The step of “providing a biological sample” may further include various isolation and/or purification steps known in the art for providing a specific component of a biological sample for use in the methods described and claimed herein.

The terms “cancer” and “cancerous” refer to or describe the physiological condition in mammals that is typically characterized by unregulated cell growth. Examples of cancer include, but are not limited to, carcinoma, lymphoma, blastoma, and sarcoma. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

The term “metastasis” as used herein will be understood to refer to the spread of cancer from a primary tumor to other parts of the body. Metastasis is a sequential, multistep process in which tumor cells detach from a primary tumor, migrate through the basement membrane and extracellular matrix, and invade the lymphatic and/or blood systems. This is followed by the establishment of secondary tumors at distant sites.

Numerous aspects and advantages of the inventive concept(s) will be apparent to those skilled in the art upon consideration of the following detailed description which provides illumination of the practice of the presently disclosed and/or claimed inventive concept(s).

The presently disclosed and/or claimed inventive concept(s) generally relates to methods of detecting and treating post-infarct myocardial remodeling and diffuse myocardial fibrosis in a patient. Such presently disclosed and/or claimed inventive concept(s) include, but are not limited to the following methods:

A method of detecting a concentration of galectin-3 in a human patient sample that is indicative of diffuse myocardial fibrosis in the human patient, the method comprising the steps of: (a) collecting a sample from a patient within seven days after an infarct episode; (b) measuring the sample to determine a concentration of a galectin-3 biomarker present in the sample; and (c) comparing the concentration of the galectin-3 biomarker to a threshold galectin-3 concentration.

The method, wherein the threshold galectin-3 concentration is 10.15 nanograms per milliliter.

The method of claim, wherein the sample comprises a serum sample.

The method, wherein the sample is collected on day 7 after an infarct episode.

The method, wherein the infarct episode comprises a ST-segment elevation myocardial infarction.

The method, wherein the concentration of the galectin-3 biomarker is measured via at least one methodology selected from the group consisting of spectrophotometery, at least one immunoassay, at least one enzyme-linked immunosorbent assay, an immunoanalyzer, and combinations thereof.

A method for diagnosing and prophylactically treating diffuse myocardial fibrosis in a post-infarct patient, the method comprising the steps of: (a) collecting a sample from a patient within seven days after an infarct episode; (b) measuring the sample to determine a concentration of a galectin-3 biomarker present in the sample; (c) comparing the concentration of the galectin-3 biomarker to a threshold galectin-3 concentration; and (d) administering an effective amount of a therapeutic composition to the patient when the concentration of the galectin-3 biomarker is equal to or greater than the threshold galectin-3 concentration.

The method, wherein the threshold galectin-3 concentration is 10.15 nanograms per milliliter.

The method, wherein the sample comprises a serum sample.

The method, wherein the sample is collected on day 7 after an infarct episode.

The method, wherein the infarct episode comprises a ST-segment elevation myocardial infarction.

The method, wherein the concentration of the galectin-3 biomarker is measured via at least one methodology selected from the group consisting of spectrophotometery, at least one immunoassay, at least one enzyme-linked immunosorbent assay, an immunoanalyzer, and combinations thereof.

The method, wherein the administration of the effective amount of the therapeutic composition is oral administration.

The method, wherein the therapeutic composition is selected from the group consisting of angiotensin receptor blockers, beta blockers, calcium channel blockers, renin-angiotensin system (RAS) inhibitors, angiotensin-converting-enzyme (ACE) inhibitors, Ang II receptor blockers (ARBs), direct renin inhibitors, and aldosterone antagonists, transforming growth factor beta (TGF-beta) inhibitors and endothelin (ET) inhibitors, histone deacetylases (HDACs) inhibitors, ivabradine, diltiazem, tadalafil, isosorbide dinitrate and hydralazine, erythropoietin, cyclosporine, thalidomide, and anti-inflammatory drugs impacting cytokines, TD139 galectin-3 inhibitor, or combinations thereof.

The method, wherein the effective amount of the therapeutic composition is administered within the first 180 days after the infarct episode.

### EXAMPLE(S)

A non-limiting Example is provided hereinbelow. However, the presently disclosed and/or claimed inventive concept(s) is to be understood to not be limited in its application to the specific experimentation, results and laboratory procedures. Rather, the Example is simply provided as one of various embodiments and is meant to be exemplary, not exhaustive.

### Example 1

### Utility of Galectin-3 in Predicting Post-Infarct Remodeling after Acute Myocardial Infarction Based on Extracellular Volume Fraction Mapping

Acute myocardial infarction triggers extracellular matrix (ECM) expansion in the remote myocardium. Myocardial extracellular volume fraction (ECV), determined by cardiovascular magnetic resonance (CMR), permits quantification of interstitial space expansion. In one aspect, the presently disclosed and/or claimed inventive concept(s) determine the relationship between early serum biomarkers with a plausible role in fibrosis generation and 180-day post-infarct remote myocardium remodeling using ECV.

In 26 patients with ST-segment elevation myocardial infarction (STEMI), functional imaging, T1 mapping, and late gadolinium enhancement were performed on a 3-T CMR scanner at baseline (days 3 to 5) and 180 days. Cardiac parameters analyzed included the ECV. Biomarkers were measured at days 1, 3, and 7 after STEMI.

The mean initial and follow-up left ventricular ejection fraction (LVEF) were 48.3±18.1% and 52.6±12.3%, respectively. Initial infarct size was 11.6±16.8% of LV mass. ECV in the remote myocardium at 180 days correlated with indexed end-systolic volume (ESVi) (r=0.4, p=0.045). Among all biomarkers, a significant correlation was observed between galectin-3 measured at day 7 and ECV at 6 months (r=0.428, p=0.037). A trend towards a direct correlation was also found for BNP (r=0.380, p=0.059). Multivariate linear regression analysis revealed that BNP and galectin-3 were independent predictors of long-term changes in ECV and explained nearly 30% of the variance in this parameter (r²=0.34; p=0.01 for both predictors). The classification and regression tree (CART) decision tree algorithm identified a galectin-3 cutoff value of 10.15 ng/mL to be the most powerful predictor of high ECV values (28.5%) at follow-up. A logistic regression model showed that galectin-3 at day 7 was an independent predictor of high ECV values at follow-up (OR: 22.51; CI: 95% 2.1-240.72; p=0.01) with 0.76 AUC (CI: 0.574-0.964; p=0.03).

Galectin-3 measured acutely after STEMI is a positive independent predictor of increased ECV at 6-month follow-up which is useful for long-term risk stratification and determination.

### Methods

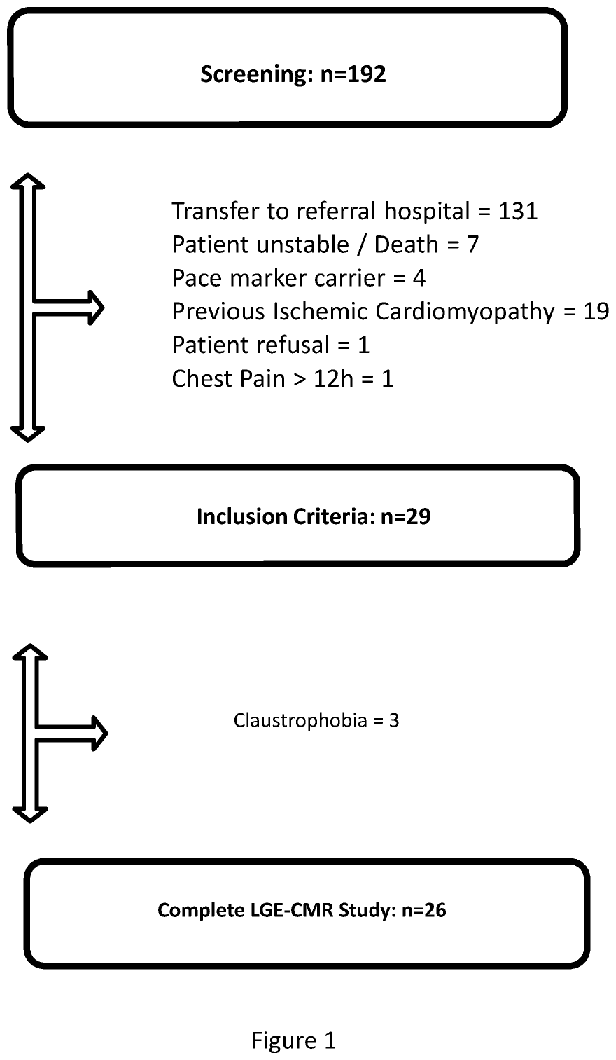

Patient Population. From June 2013 to September 2014, patients presenting with a first STEMI to the Coronary Care Unit were screened. Patients with early death and those requiring mechanical ventilation or with hemodynamic instability were not included in the study. From 192 patients screened, a total of 29 patients were included. Of 29 patients, 26 underwent a complete initial and follow-up CMR study. FIG. 1 graphically depicts a flow chart that details the selection process for patient inclusion in the study.

Primary percutaneous intervention was the reperfusion treatment, delivered by experienced on-call interventional cardiologists following unfractionated heparin, aspirin, and a loading dose of clopidogrel. At the physician's discretion and unless contraindicated, captopril or enalapril (at least 6.25 mg every 8 hours or 2.5 mg every 12 hours, respectively) and beta-blockers were initiated early, within 24 hours from admission. Serum troponin I was measured over 48 hours: every 6 hours during the first 12 hours and every 12 hours thereafter. Primary patient exclusion criteria included, but were not limited to, the presence of other underlying fibrotic pathologies affecting major organs such as liver, kidney, and lung, as these processes affect the quantification of cardiac biomarkers for extracellular matrix remodeling and fibrosis. In addition, patients with a previous history of renal or liver failure were also excluded from the study.

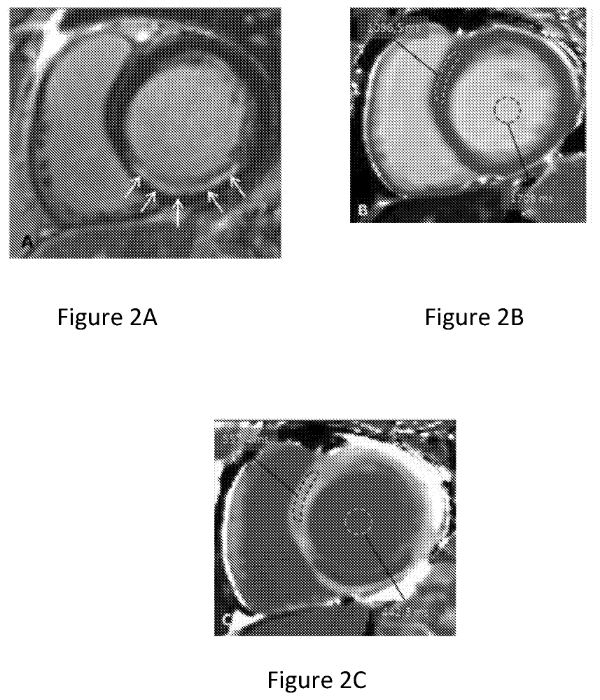

CMR Protocol and Analysis. CMR examinations were performed at baseline (days 3 to 5) and 180 days after STEMI. CMR images were acquired with a 3-T system (MAGNETOM Trio™, a Tim® System, commercially offered by Siemens Healthcare, Erlangen, Germany) using electrocardiographic triggering and a 32-channel phased array cardiovascular coil. The sequences used in cine image acquisition inversion-recovery imaging after gadolinium administration, and in T1 mapping, together with the techniques used in measuring LV volumes, LVEF, ECV, and myocardial infarct size (14) are commonly described and known in the art. FIGS. 2A-2C show quantification of the extracellular volume fraction (ECV) from pre- and post-contrast T1 maps in a patient with percutaneous coronary intervention 180 days prior for right coronary occlusion. FIG. 2A shows a T1 map of a late gadolinium enhancement image showing an inferior scar. FIG. 2B shows a T1 map in which the ECV was calculated with regions drawn pre-injection of the gadolinium contrast agent. FIG. 2C shows a T1 map in which the ECV was calculated with regions drawn 15 minutes post-injection of the gadolinium contrast agent. With respect to FIGS. 2A-2C, the ECV was calculated with the equation: ECV=(1-Hematocrit)×(ΔR1_{myocardium}/ΔR1_{blood}), wherein ΔR1 is defined as (1/T1_{pre-gandolinium contrast}−1/T1_{post-gadolinium} contrast). For the particular patient shown in FIGS. 2A-2C, the Hematocrit was 44% and the ECV in the remote myocardium was calculated to be 29.5% (which is slightly elevated, reflecting diffuse fibrosis).

Laboratory Measurements. Blood samples were obtained from study participants at days 1, 3, and 7 after STEMI. Samples were collected via methods commonly known in the art, including, but not limited to, via needle(s) followed by subsequent dispensing of the sample into at least one collection vessel. Briefly, brain natriuretic peptide (BNP), Enhanced Liver Fibrosis score (ELF™, calculated from the measurement of PIIINP, HA, and TIMP-1), troponin I (TnI), dehydroepiandrosterone sulfate (DHEA-504), insulin-like growth factor 1 (IGF-1), tumor necrosis factor α (TNF-α), pregnancy-associated plasma protein (PAPP-A), the receptor of the cytokine interleukin 2 (IL2R), IL-1E, IL-6, IL-8, IL-10, beta-C-terminal telopeptide (CITP), C-terminal propeptide of type I collagen (PICP), matrix metalloproteinase-1 (MMP-1), MMP-2, MMP-9, apelin, and galectin-3 were quantified in accordance with methods and procedures commonly known in the art, including, but not limited to, via mass spectrometry, microarray assays. The intra- and inter-assay coefficients of variation were lower than 5% and 10%, respectively, in all cases. Other biochemical and hematological parameters were measured by standard procedures, including, but not limited to, standard procedures at the Core Laboratory of the Biomedical Diagnostic Centre of the Hospital Clinic of Barcelona.

Statistical Analysis. Normal QQ plot and the Shapiro-Wilk test were used to identify non-normally distributed variables. Normally distributed data were expressed as mean values±S.D. Non-normally distributed data were expressed as medians±interquartile range. CMR measurements were adjusted for total body surface area. The strength of the relationship between the laboratory parameters at days 1, 3, and 7 post-STEMI and the ECV at day 180 was assessed using the Pearson and Spearman correlation coefficients. Correlations between ECV at day 180 and clinical characteristics—including, but not limited to, age, body mass index, total cholesterol level, and blood pressure—were also conducted. The statistical analysis between ECV and categorical clinical data—including, but not limited to, hypertension, smoking habit, diabetes, dyslipidemia, Killip class—was performed using a two-sided Student's t-test or analysis of variance for independent samples (with Tukey's post hoc test), when appropriate. The parameters that showed a near-significant or significant correlation with ECV (BNP and galectin-3) were selected for multivariate statistical analysis, considering continuous or categorized ECV as the response variable. The multivariate statistical analyses conducted were classification and regression tree (CART) algorithm, lineal regression, and logistic regression. In the case of linear regression and logistic regression, the validity of the models was assessed by plotting the residuals or the Pearson residuals against fitted values and by plotting the influence of each observation on the fitted response according to the Cook's distance. A multivariable logistic regression model adjusted for baseline covariates of galectin-3, age, diabetes, creatinine, and BNP was also assessed. Sensitivity, specificity, positive predictive value, and negative predictive value were also calculated to evaluate the performance of galectin-3 for ECV prediction at day 180 post-STEMI. All the statistical analyses were performed using public libraries from the Comprehensive R Archive Network (CRAN which can be found at the following URL: http://CRAN.R-project.org) rooted in the open source statistical computing environment R, version 3.1 (available at the following URL: http://www.R-project.org/). A p-value less than 0.05 was considered statistically significant.

### Results

Patient Characteristics. Among the 26 patients, 24 (92%) were male with a mean age of 58.3±11.4 years. Most patients (88.5%) were in Killip class I. Table 1 hereinbelow summarizes the demographic and clinical characteristics of the patients at admission and during the follow-up.

TABLE 1
Association of ECV at day 180 after STEMI with
demographic and clinical characteristics of patients
(n = 26) at baseline and during the follow-up

| | | | |
|---|---|---|---|
| Days | Mean ± s.d | Correla- | |
| after | median | tion coef- | p- |
| STEMI | (25-75 IQR) | ficient (r) | value |

| | | | |
|---|---|---|---|
| Demographic | | | |
| characteristics | | | |
| Age (years) | 58.3 ± 11.4 | −0.05 | 0.792 |
| Sex (male, %) | 92.3 | | 0.075 |
| BMI (kg/m²) | 27.1 ± 3.1 | −0.28 | 0.169 |
| Heart rate (bpm) | 71.1 ± 18.8 | 0.32 | 0.116 |
| Systolic blood | 138.5 ± 28.0 | −0.06 | 0.769 |
| pressure (mmHg) | | | |
| Diastolic blood | 85.9 ± 18.0 | −0.03 | 0.880 |
| pressure (mmHg) | | | |
| Clinical history | | | |
| Hypertension | 15.4 | | 0.058 |
| (Yes, %) | | | |
| Smoking | 53.8 | | 0.112 |
| (Yes, %) | | | |
| Diabetes mellitus | 7.7 | | 0.255 |
| (Yes, %) | | | |
| Dyslipidemia | 26.9 | | 0.570 |
| (Yes, %) | | | |
| Killip class | I: 88.5 | | 0.389 |
| (Yes, %) | IV: 11.5 | | |

Abbreviations and Description of Table 1:
IQR, interquartile range;
BMI, body mass index.
Normally distributed data are expressed as mean values ± S.D. The correlation between demographic and clinical parameters at 1, 3, 7, and 180 days post-STEMI and the ECV at day 180 was assessed using the Pearson correlation test. The significance of association between ECV at day 180 and the categorical and ordinal variables was assessed by unpaired Student's t-test or the ANOVA test (with Tukey's post hoc test), respectively.

At admission, all the patients had a positive TnI value with a median of 78.7 ng/mL (IQR 105.5). The infarct-related artery was the left anterior descending artery in 12 (46%) cases, followed by 10 (39%) and 4 (15%) the right and left circumflex artery, respectively. The average infarct size was 11.6±16.8% of LV mass.

CMR Imaging Findings at Follow-Up. ECV quantification of remote myocardium at 180 days was 27.9±2.8% and correlated with ESVi (r=0.397, p=0.044). In contrast, neither infarct size, whether acute (r=0.042, p=0.85) or chronic (r=−0.075, p=0.74), nor EDVi (r=0.35, p=0.08) or LV EF (r=−0.191, p=0.35) correlated with ECV at 180 days.

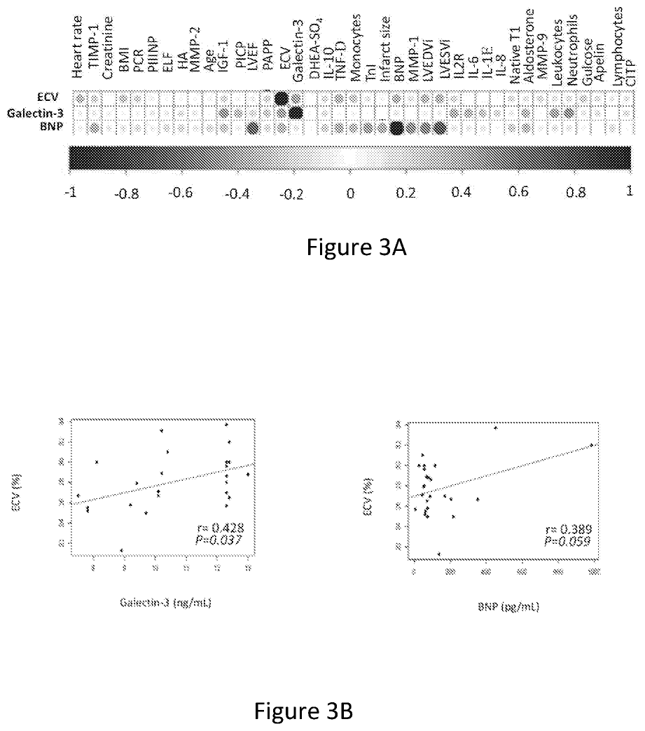

Predictors of ECV. 26 biochemical and 4 hematological parameters were measured in the studied population at the early time points of 1, 3, and 7 days after STEMI. For each parameter, only the value of the time point that showed the highest correlation coefficient with the response variable ECV were considered for statistical analysis. This statistical analysis information is shown hereinbelow in Table 2. Among all the biochemical parameters and as shown in FIGS. 3A and 3B, a positive trend between ECV at 6 months and early-time measurements of BNP (r=0.38, p=0.059) and a significant correlation with galectin-3 at 7 days was found (r=0.428, p=0.037). Co-linearity was not observed between galectin-3 and BNP. Galectin-3 significantly correlated with the circulating levels of neutrophils (r=0.48, p=0.02), leukocytes (r=0.42, p=0.04), and IGF-1 (r=−0.45, p=0.03). A positive trend with aldosterone was also observed (r=0.36, p=0.08) (FIG. 3A). In contrast, BNP significantly correlated with TIMP-1 (r=0.42, p=0.04), TnI (r=0.52, p=0.007), and monocytes (r=0.53, p=0.006).

TABLE 2
Association of ECV at Day 180 After STEMI with
CRM and Laboratory Characteristics of Patients
(n = 26) at Baseline and During the Follow-Up

| | Days | Mean + s.d. | | Correla- | |
|---|---|---|---|---|---|
| | after | median | | tion coef- | p- |
| | STEMI | (25-75 IQR) | | ficient (r) | value |
| CMR | | | | | |
| measurements | | | | | |
| LVEF (%) | 7 | 48.3 ± 18.1 | | −0.09 | 0.662 |
| LVEF (%) | 180 | 52.6 ± 12.3 | | −0.19 | 0.349 |
| Native T1 (ms) | 7 | 1036.3 ± 50.7 | | 0.13 | 0.534 |
| Native T1 (ms) | 180 | 1036.0 ± 69.9 | | 0.23 | 0.910 |
| LVEDVi | 7 | 88.7 ± 23.4 | | 0.34 | 0.095 |
| (mL/m²) | | | | | |
| LVEDVi | 180 | 91.1 ± 25.3 | | 0.34 | 0.084 |
| (mL/m²) | | | | | |
| LVESVi | 7 | 44.7 ± 16.4 | | 0.36 | 0.762 |
| (mL/m²) | | | | | |
| LVESVi | 180 | 44.6 ± 8.9 | | 0.40 | 0.045* |
| (mL/m²) | | | | | |
| Infarct size (%) | 7 | 11.6 ± 16.8 | | −0.12 | 0.568 |
| Infarct size (%) | 180 | 10.2 ± 12.2 | | −0.12 | 0.581 |
| Laboratory and | | | | | |
| hemodynamic | | | | | |
| parameters | | | | | |
| BNP (pg/mL) | 7 | 72.04 | (98.90) | 0.38 | 0.059 |
| ELF | 7 | 9.23 ± 0.78 | | 0.04 | 0.852 |
| PIIINP (ng/mL) | 7 | 7.03 ± 2.33 | | −0.07 | 0.740 |
| HA (μg/mL) | 7 | 50.42 | (49.55) | 0.10 | 0.644 |
| TIMP-1 (ng/mL) | 7 | 275.19 ± 68.94 | | −0.222 | 0.286 |
| Tnl (ng/mL) | peak | 78.66 | (105.51) | −0.132 | 0.519 |
| DHEA-SO₄ | 3 | 111.80 ± 55.87 | | −0.005 | 0.982 |
| (μg/mL) | | | | | |
| IGF-1 (ng/mL) | 3 | 201.61 ± 75.73 | | −0.204 | 0.351 |
| TNF-α (pg/mL) | 3 | 9.47 | (3.50) | 0.366 | 0.130 |
| PAPP-A (mU/mL) | 7 | 0.00 | (0.01) | −0.248 | 0.254 |
| IL2R (pMol) | 3 | 586.00 | (240.00) | −0.16 | 0.466 |
| IL-1β (pg/mL) | 3 | 0.56 | (0.68) | 0.08 | 0.725 |
| IL-6 (pg/mL) | 3 | 9.260 | (14.75) | 0.11 | 0.627 |
| IL-8 (pg/mL) | 3 | 11.00 | (17.64) | 0.16 | 0.472 |
| IL-10 (pg/mL) | 3 | 1.57 | (1.30) | 0.03 | 0.874 |
| CITP (ng/mL) | 7 | 0.29 | (0.22) | 0.12 | 0.579 |
| PICP (ng/mL) | 7 | 90.00 | (45.00) | 0.22 | 0.284 |
| MMP-1 (ng/mL) | 7 | 5.90 | (9.47) | 0.14 | 0.527 |
| MMP-9 (ng/mL) | 7 | 196.45 | (267.85) | −0.26 | 0.217 |
| MMP-2 (ng/mL) | 7 | 205.35 ± 43.92 | | 0.039 | 0.857 |
| Apelin (pg/mL) | 3 | 0.27 | (0.13) | 0.16 | 0.450 |
| Galectin-3 (ng/mL) | 7 | 10.654 ± 1.81 | | 0.428 | 0.037* |
| Aldosterone (ng/dL) | 3 | 7.15 | (9.025) | 0.14 | 0.501 |
| Creatinine (mg/dL) | 1 | 0.90 | (0.30) | 0.05 | 0.80 |
| CRP (mg/dL) | 1 | 1.54 | (3.53) | −0.12 | 0.58 |
| Leukocytes | 3 | 8.69 ± 1.50 | | 0.13 | 0.525 |
| (10⁹ cells/L) | | | | | |
| Neutrophils | 3 | 5.87 ± 1.33 | | 0.15 | 0.456 |
| (10⁹ cells/L) | | | | | |
| Lymphocytes | 3 | 1.80 | (0.40) | −0.022 | 0.915 |
| (10⁹ cells/L) | | | | | |
| Monocytes | 3 | 0.60 | (0.325) | 0.05 | 0.810 |
| (10⁹ cells/L) | | | | | |

| | | | | |
|---|---|---|---|---|
| Glucose (mg/dL) | 1 | 132.63 ± 58.37 | 0.25 | 0.223 |

Abbreviations and Description of Table 2:
IQR, interquartile range;
*p-value, significance.
Normally distributed data are expressed as mean values ± S.D. and non-normally distributed data are expresses as medians (IQR). The correlation between demographic, clinical, and CRM/laboratory parameters at days 1, 3, 7, and 180 post-STEMI and the ECV at day 180 was assessed using the Pearson correlation test (for normally distributed data) and the Spearman correlation coefficient (for non-normally distributed data).

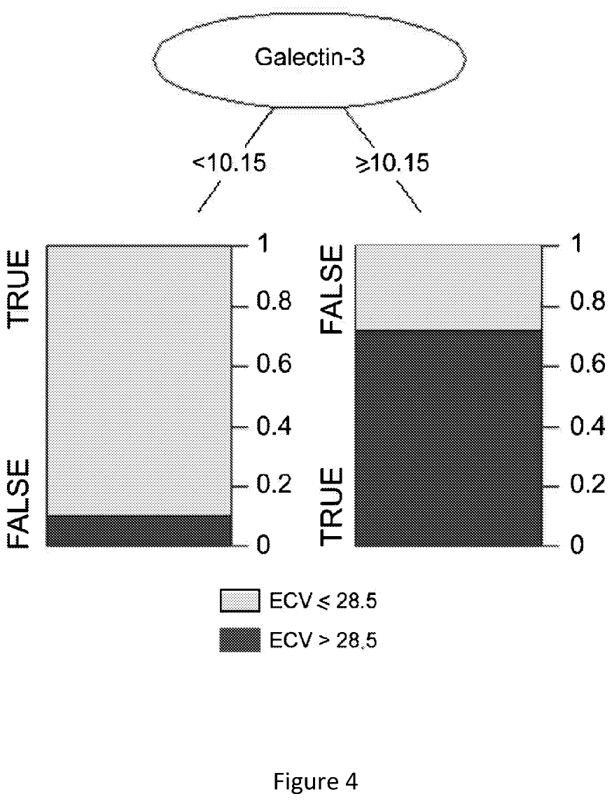

A non-significant correlation between BNP and MMP-1 was also found (r=0.39, p=0.081) (as shown in FIG. 3A). Both BNP and galectin-3 were included in the multivariate linear regression analysis, which showed that both parameters were positive independent predictors of ECV at 180 days after STEMI (coefficient for galectin-3: 0.61±0.29, p=0.047; coefficient for BNP: 0.01±0.003, p=0.043) and explained 34% of the variance in ECV (r2=0.34; F-statistic: 5.14; p=0.01). Next, the ECV values obtained in the patient population were categorized according to previous studies that estimated an optimal cutoff of 28.5% for “elevated” ECV. Applying this categorization to the ECV variable, mean concentrations of galectin-3 were significantly higher among patients who showed elevated ECV than in those with an ECV index≤28.5 (11.4±0.5 vs. 9.9±0.4, respectively; p<0.05). In addition, galectin-3 but not BNP maintained its predictive value in multivariate statistical models when ECV was transformed into a categorical variable. For instance, a CART decision tree considering both BNP and galectin-3 as explanatory variables yielded galectin-3 as the most powerful predictor to stratify patients with higher ECV values versus low ECV values. As shown in FIG. 4, the optimal cutoff value/threshold for galectin-3 generated by the CART algorithm was 10.15 ng/mL. This value, which was similar to the mean value obtained in our population (10.60 ng/mL), yielded a sensitivity of 69.2% and a specificity of 90.9% for identifying patients with elevated ECV. This optimal cutoff value was also used to build a logistic regression model to predict the categorical outcome of elevated ECV. In agreement with the CART model, categorized galectin-3 was a positive independent predictor of high ECV after long-term follow-up (OR: 22.5; CI: 95% 2.1-240.7; p=0.01), thus providing evidence of the discriminatory ability of galectin-3. The ROC analysis for elevated ECV showed an AUC for the predicted values of the logistic model of 0.76 (CI: 0.57-0.96; p=0.03). The sensitivity and specificity of the logistic model were equivalent to the values obtained using the CART algorithm.

Table 3 hereinbelow shows the adjusted odds for galectin-3 occurrence of elevated ECV at 180 days post STEMI after correction for established risk factor of heart failure. In the adjusted model, galectin-3 was the only significant predictor of elevated ECV despite the inclusion of age, diabetes, creatinine, and BNP. In addition, both the unadjusted and the adjusted models showed similar statistical estimates for galectin-3, ruling out the involvement of confounding variables in the result of the logistic regression model.

TABLE 3
Unadjusted and Adjusted Odd Ratios for Elevated ECV

| | Unadjusted | | | Adjusted | | |
|---|---|---|---|---|---|---|
| | Odds | | | Odds | | |
| [symbol] | Ratio | 95% CI | P | Ratio | 95% CI | P |
| [symbol] | 22.5 | 2.1-240.7 | 0.01* | 22.2 | [symbol] | 0.02* |
| Age | | | | 0.9 | [symbol] | 0.92 |
| [symbol] | | | | | | |
| Diabetes | | | | 0.5 | [symbol] | 0.42 |
| [%] | | | | | | |
| Creatinine | | | | 0.07 | [symbol] | 0.52 |
| (mg/dL) | | | | | | |
| BNP | | | | 1.0 | 0.9-1.0 | [symbol] |
| ( [symbol] g/mL) | | | | | | |

Abbreviations and Description of Table 3: *p-value, significance; odds ratios, 95% CIs, and P values for predictors of elevated ECV at 180 days after STEMI. Results from unadjusted regression logistic model (columns 2 to 4) and results from the adjusted regression logistic model (columns 5 to 7) are shown. [symbol]
indicates data missing or illegible when filed

### Discussion

Example 1 provides evidence that demonstrates that galectin-3, measured in serum early after STEMI, is independently associated with and indicative of increased ECV in the remote non-infarcted myocardium, which is itself indicative of diffuse myocardial fibrosis (DMF).

Characterization of Diffuse Myocardial Fibrosis with CMR T1 Mapping. LV remodeling after myocardial infarction is defined in terms of infarct size and severity, location, and its subsequent impact on the function, shape, and size of the left ventricle. The mechanism underlying LV remodeling after myocardial infarction involves not only the infarcted myocardium, but also changes in the adjacent and remote myocardium. The remodeling of remote myocardium is a well-known process in chronic ischemic disease and involves diffuse fibrosis. Although previously thought to be a completely normal region immediately after myocardial infarction, acute changes occur in the remote myocardium consisting of ECM expansion due to diffuse fibrosis. In this regard, a recent study demonstrated impaired systolic LV wall thickness early in STEMI patients involving the non-infarcted remote myocardium, underlying the contribution of non-ischemic myocardium in LV dysfunction. Current T1 myocardial mapping techniques offer a quantitative assessment of the tissue composition on a voxel-by-voxel basis. Both native and post-contrast T1 mapping, including ECV mapping, reflect the presence of DMF on remote myocardium. Native T1 mapping, before the administration of gadolinium-based contrast agents (GBCAs), comprises myocardial signal from both the intracellular and extracellular spaces and has been shown to be elevated in a variety of pathologies where increased water is present, such as edema and focal or diffuse fibrosis. Nevertheless, after administration of gadolinium-based contrast, the signal from the extracellular space dominates. If the ratio of signal change in blood and myocardium after contrast administration is calculated, corrected by the hematocrit once an equilibrium between plasma and myocardial contrast agent concentration has been reached, the ECV can be calculated. Several studies have demonstrated the usefulness of ECV for characterization of myocardial disease, such as valve disease, cardiomyopathies, and infiltrative diseases. ECV has also been associated with increased mortality. In ischemic disease, diffuse fibrosis represents an additional mechanism to the traditional criterion of LV remodeling after myocardial infarction, together with the presence of an infarct scar and altered LV pressure and volume, leading to LV dilation and progressive dysfunction. In this regard, a recent study found subtle ECV alterations in myocardium remote from regions of infarct consistent with diffuse myocardial fibrosis, these changes being related to the severity of LV dysfunction. These findings are consistent with histologically detected changes in ECM at sites distant from myocardial scarring.

In the present study, no correlation was found between infarct size and ECV at follow-up. It has been proposed that changes in ECM in remote myocardium account for approximately two-thirds of all cardiac fibrous tissue and are supposed to be the primary component of ventricular remodeling in ischemic cardiomyopathy. However, these changes develop progressively and are apparent at time intervals longer than 6 months after follow-up. In the current study, it was found that ECV correlated with ESVi at 180 days, a result that is consistent with post-infarct remodeling and with prior studies.

Galectin-3 in Ventricular Remodeling. Galectin-3 is a 30 kDa member of the galectins family that in human patients is encoded by the gene LGALS3. Several studies have demonstrated the pro-inflammatory role of galectin-3, mainly as a result of the attraction and/or activation of monocytes and macrophages. In addition to this pro-inflammatory role, galectin-3 is also required for TGF-beta-mediated myofibroblast activation, proliferation, and ECM production. In the context of heart disease, galectin-3 has been shown to be overexpressed early in the infarcted myocardium in mice and in the hypertrophic heart of rats overexpressing the Ren-2d renin gene that later developed heart failure. In a recent study, it has been demonstrated that an intrapericardial infusion of low-dose galectin-3 in healthy rats led to left ventricular dysfunction and a significant increase of collagen I. Another recent study implicated galectin-3 in the pathogenesis of cardiac fibrosis in mice, demonstrating that genetic disruption and pharmacological inhibition of galectin-3 blunted cardiac fibrosis and LV dysfunction in experimental models of cardiac remodeling. These preclinical studies demonstrated that galectin-3 plays a major role in the development of cardiac fibrosis. This link, however, is not well established in the clinical setting, most likely due to the ethical constraints associated with performing invasive assessment of fibrosis in patients for research purposes. Nevertheless, several publications suggest that the pro-fibrotic role of galectin-3 may also occur in human patients. For instance, galectin-3 correlated significantly with certain biomarkers involved in ECM turnover in patients early after STEMI and in patients with chronic heart failure. Galectin-3 has also been shown to be an independent predictor of left ventricular remodeling and mortality in patients with chronic heart failure. The applicability of galectin-3 for diagnosis or prognosis in either acute or chronic heart failure has been validated in several human studies. In this context, the 2013 ACCF/AHA Guideline for the management of heart failure considered galectin-3 predictive of hospitalization/death and useful for additive risk stratification of patients with acute or chronic heart failure. While these studies provided clinical evidence about the diagnostic and prognostic value of galectin-3 in heart failure, all of them lack proof of the direct association of galectin-3 and myocardial fibrogenesis. Recently, the significant association of galectin-3 and CE-CMR-assessed fibrosis in patients with non-ischemic dilated cardiomyopathy has been reported. In contrast to the previous publications, a recent study demonstrated that galectin-3 is a true biomarker of myocardial fibrosis. However, the causative link of galectin-3 and fibrosis was unexplored in this recent study.

Accordingly, in the present study, galectin-3 levels taken early after STEMI were correlated with the direct assessment of myocardial fibrosis at 180 days after STEMI. This strategy enabled the identification of galectin-3 as the only independent predictive factor for the development of late myocardial fibrosis, even in the presence of troponin and BNP as explanatory variables. As shown in the present study, the predictive value of galectin-3 was also conserved after correction for established risk factors for heart failure such as age, BNP level, renal function, and diabetes mellitus. In addition, the levels of galectin-3 early after STEMI correlated with the co-expression of inflammatory components and showed a substantially significant correlation trend with aldosterone. Both inflammatory factors and aldosterone have previously been described as strong inductors of galectin-3 release. Accordingly, as a result of the present study, targeting the renin-angiotensin-aldosterone system early after STEMI blocks galectin-3 overexpression and, as a result, ameliorates, mitigates, and therapeutically treats cardiac remodeling and diffuse myocardial fibrosis.

### Clinical Relevance

Integration of serum biomarkers with those derived from CMR imaging techniques represents an innovative and effective method for the accurate diagnosis of the remodeling process after myocardial infarction.

The present study confirms a pro-fibrotic effect of galectin-3 in human myocardium after STEMI, indicating the utility of this biomarker in the prediction of myocardial remodeling. This mediator, together with the information obtained from the CMR T1 mapping techniques, confirmed the existence of a continuous process of myocardial fibrosis after myocardial infarction, even in its early stages, and provided important information regarding patient management. Galectin-3 early identifies patients at high risk who will benefit from drug therapies that prevent remodeling.

### CONCLUSIONS

Circulating serum galectin-3 biomarker early after STEMI is an independent predictor of LV remodeling, as assessed with ECV derived from CMR. The present study is the first study to report the correlation of a serum biomarker with ECV in survivors of acute myocardial infarction that confirms that pathological role of galectin-3 in fibrogenic processes and cardiac remodeling.

Thus, in accordance with the presently disclosed and/or claimed inventive concept(s), there has been provided method(s) for detecting and mitigating post-infarct myocardial remodeling that fully satisfies the objectives and advantages set forth hereinabove. Although the presently disclosed and/or claimed inventive concept(s) has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and/or claimed inventive concept(s).

What is claimed is:

1. A method of detecting a concentration of galectin-3 in a human patient sample that is indicative of diffuse myocardial fibrosis in the human patient, the method comprising the steps of:
(a) collecting a sample from a patient within seven days after an infarct episode;
(b) measuring the sample to determine a concentration of a galectin-3 biomarker present in the sample; and
(c) comparing the concentration of the galectin-3 biomarker to a threshold galectin-3 concentration.

2. The method of claim 1, wherein the threshold galectin-3 concentration is 10.15 nanograms per milliliter.

3. The method of claim 1, wherein the sample comprises a serum sample.

4. The method of claim 1, wherein the sample is collected on day 7 after an infarct episode.

5. The method of claim 1, wherein the infarct episode comprises a ST-segment elevation myocardial infarction.

6. The method of claim 1, wherein the concentration of the galectin-3 biomarker is measured via at least one methodology selected from the group consisting of spectrophotometery, at least one immunoassay, at least one enzyme-linked immunosorbent assay, an immunoanalyzer, and combinations thereof.

7. A method for diagnosing and prophylactically treating diffuse myocardial fibrosis in a post-infarct patient, the method comprising the steps of:
(a) collecting a sample from a patient within seven days after an infarct episode;
(b) measuring the sample to determine a concentration of a galectin-3 biomarker present in the sample;
(c) comparing the concentration of the galectin-3 biomarker to a threshold galectin-3 concentration; and
(d) administering an effective amount of a therapeutic composition to the patient when the concentration of the galectin-3 biomarker is equal to or greater than the threshold galectin-3 concentration.

8. The method of claim 7, wherein the threshold galectin-3 concentration is 10.15 nanograms per milliliter.

9. The method of claim 7, wherein the sample comprises a serum sample.

10. The method of claim 7, wherein the sample is collected on day 7 after an infarct episode.

11. The method of claim 7, wherein the infarct episode comprises a ST-segment elevation myocardial infarction.

12. The method of claim 7, wherein the concentration of the galectin-3 biomarker is measured via a methodology selected from the group consisting of spectrophotometery, at least one immunoassay, at least one enzyme-linked immunosorbent assay, an immunoanalyzer, and combinations thereof.

13. The method of claim 7, wherein the administration of the effective amount of the therapeutic composition is oral administration.

14. The method of claim 7, wherein the therapeutic composition is selected from the group consisting of angiotensin receptor blockers, beta blockers, calcium channel blockers, renin-angiotensin system (RAS) inhibitors, angiotensin-converting-enzyme (ACE) inhibitors, Ang II receptor blockers (ARBs), direct renin inhibitors, and aldosterone antagonists, transforming growth factor beta (TGF-beta) inhibitors and endothelin (ET) inhibitors, histone deacetylases (HDACs) inhibitors, ivabradine, diltiazem, tadalafil, isosorbide dinitrate and hydralazine, erythropoietin, cyclosporine, thalidomide, and anti-inflammatory drugs impacting cytokines, TD139 galectin-3 inhibitor, or combinations thereof.

15. The method of claim 7, wherein the effective amount of the therapeutic composition is administered within the first 180 days after the infarct episode.
